(19) United States
(12) Patent Application Publication
(10) Pub. No.: US 2017/0290872 A1
(43) Pub. Date: Oct. 12, 2017

(54) ORAL SPRAY CONTAINING IXERIS DENTATA NAKAI EXTRACT AS ACTIVE INGREDIENT FOR PREVENTING OR TREATING XEROSTOMIA CAUSED BY DIABETES

(71) Applicants: INDUSTRIAL COOPERATION FOUNDATION CHONBUK NATIONAL UNIVERSITY, Jeonju-si (KR); Daegu Gyeongbuk Institute of Science and Technology, Dalseong-gun (KR); REPUBLIC OF KOREA (MANAGEMENT: RURAL DEVELOPMENT ADMINISTRATION), Jeonju-si (KR); INDUSTRY-ACADEMIC COOPERATION FOUNDATION, YONSEI UNIVERSITY, Seoul (KR)

(72) Inventors: Han Jung CHAE, Jeonju-si (KR); Hyung-Ryong KIM, Jeonju-si (KR); Seung Hyun KIM, Incheon (KR); Sang Won LEE, Yuseong-gu (KR)

(73) Assignees: INDUSTRIAL COOPERATION FOUNDATION CHONBUK NATIONAL UNIVERSITY, Jeonju-si (KR); Daegu Gyeongbuk Institute of Science and Technology, Dalseong-gun (KR); REPUBLIC OF KOREA (MANAGEMENT: RURAL DEVELOPMENT ADMINISTRATION), Jeonju-si (KR); INDUSTRY-ACADEMIC COOPERATION FOUNDATION, YONSEI UNIVERSITY, Seoul (KR)

(21) Appl. No.: 15/440,837

(22) Filed: Feb. 23, 2017

(30) Foreign Application Priority Data
Feb. 23, 2016 (KR) 10-2016-0021549
Feb. 23, 2017 (KR) 10-2017-0023894

(51) Int. Cl. A61K 36/28; A61K 8/97; A61Q 11/00; A61K 9/00

(52) U.S. Cl. CPC A61K 36/28; A61K 9/006; A61K 8/97; A61Q 11/00; A61K 2236/33; A61K 2236/39; A61K 2236/53

(57) ABSTRACT

An oral spray formulation using an Ixeris dentata Nakai extract as an active ingredient of a composition for preventing and treating xerostomia occurring as a diabetic complication, particularly an oral spray formulation capable of administering a drug through a simple and rapidly absorbable route is provided. The secretion of saliva is reduced as cells from a submandibular gland tissue of a diabetes model animal are destroyed. However, when the Ixeris dentata Nakai extract of the present invention is sublingually sprayed into a sublingual region of the diabetes model animal, expression levels of α-amylase, AQP5 and NHE1 can significantly increase, resulting in an increased secretion quantity and rate of saliva.

When the Ixeris dentata Nakai extract is administered using the formulation of the present invention, the Ixeris dentata Nakai extract is sublingually sprayed through the spray formulation, and thus can be directly locally administered to a salivary gland tissue having an activity to induce secretion of saliva. Therefore, saliva secretion can be induced by rapid absorption of the active ingredient of the Ixeris dentata Nakai extract in the salivary gland tissue without any absorption process through the digestive system, resulting in minimized side effects.

### CROSS-REFERENCE TO RELATED APPLICATION

This application claims priority to and the benefit of Korean Patent Application No. 2016-0021549, filed on Feb. 23, 2016 and Korean Patent Application No. 2017-0023894, filed on Feb. 23, 2017, the disclosure of which is incorporated herein by reference in its entirety.

### BACKGROUND

### 1. Field of the Invention

The present invention relates to a use of an Ixeris dentata Nakai extract as an active ingredient of a composition for preventing and treating xerostomia occurring as a diabetic complication, and more particularly, to an oral spray formulation capable of administering a drug through a simple and rapidly absorbable route.

### 2. Discussion of Related Art

Xerostomia is a symptom in which the mouth becomes dry since saliva is not continuously secreted due to certain causes, and is known to develop due to pathological atrophy and aging of salivary glands. A variety of causes of xerostomia have been reported. In this case, the quantity of saliva may be reduced due to systemic causes such as Sjogren's syndrome, anemia, diabetes, nutrient deficiency, aging, etc. In addition, a decrease in the quantity of saliva may also be caused due to various medications, neurological diseases, etc.

Saliva is secreted from major large salivary glands such as a parotid gland, a submandibular gland, and a sublingual gland, and minor salivary glands which are uniformly distributed in the mouth. When the function of such a tissue is deteriorated, the secretion of saliva is reduced, resulting in symptoms of xerostomia. When the mouth dryness occurs, an effect of salivary fluid, proteins and mucin on mouth protection may be remarkably reduced, resulting in destroyed salivary glandular tissue. When the secretion of saliva is reduced and chronic xerostomia is developed, articulatory changes occur, and it is difficult to chew and swallow foods, resulting in burning mouth syndrome and increased risk of dental cavities and oral diseases.

Intracellular factors having an influence on saliva secretion include digestive enzymes such as amylases, and channel proteins such as aquaporin 5 (AQPS). The secretion of saliva may be promoted by agonists including electrolytes such as a muscarinic agonist or an adrenergic agonist. α-Amylase is a representative digestive enzyme that can be found in saliva, and plays a role in early starch degradation. α-amylase is expressed in organelles which participate in the secretion of proteins in murine pancreatic acinar cells, or expressed in acinar cell and duct cells of the salivary glands, and thus may be used as a desirable marker capable of determining the function of salivary glands. AQPS is a major transport protein that is expressed in a dead-end membrane of an acinar cell secreting saliva. AQPS serves to specifically transport water, and thus plays an essential role in secretion of water that accounts for 99% of the saliva. It was reported that AQPS is abnormally expressed in non-obese diabetic mice, and mice suffering from an autoimmune disease such as Sjogren's syndrome, which is considered to be a major cause of xerostomia.

In recent years, much research has been conducted to treat xerostomia, but it is difficult to perform fundamental healing, and conventional treatment methods have been often performed. The mouth is kept moist by increasing water intake, and it is possible to chew a piece of sugar-free gum, take a lemon-flavored drink, use artificial saliva, and use a gargle solution supplemented with citric acid, etc. In addition, newly developed saliva preparations for spray and electrical stimulation methods have been used in trials, but has transient and restricted effects. Therefore, there has been research conducted to develop a therapeutic agent for xerostomia. In recent years, among chemical drugs, anethole trithione known as a muscarinic receptor agonist, and cevimeline hydrochloride have been used as salivators, but have drawbacks in that their effects are not stable, and they possibly have side effects in the digestive system, such as nausea, vomiting, anepithymia, abdominal discomfort, etc. Under these circumstances, there is a demand for development of novel therapeutic agents to treat hyposalivation.

Ixeris dentata Nakai (the family Asteraceae) is a medicinal plant that generally grows in Korea, Japan, and China, and has been used to treat various diseases such as hepatitis, dyspepsia, diabetes, allergies, and cancer. Previous reports showed that a methanol extract of Ixeris dentata Nakai has a therapeutic effect on diabetes by reducing levels of triglycerides and cholesterol in a diabetes animal model. In addition, it was reported that the antioxidant activity of Ixeris dentata Nakai reduces oxidative stress by maintaining a constant concentration of glutathione in a mouse brain.

In connection with a therapeutic effect of Ixeris dentata Nakai on xerostomia, it is known that Ixeris dentata Nakai has a therapeutic effect on xerostomia since the secretion of amylases increases when salivary gland cells are exposed in vitro to a high concentration of glucose. However, the Ixeris dentata Nakai extract has a therapeutic effect only when the Ixeris dentata Nakai extract is administered through a route of oral administration. Therefore, the Ixeris dentata Nakai extract has transient and restricted effects like therapeutic agents that have been studied in the prior art because the Ixeris dentata Nakai extract can have a therapeutic effect on xerostomia only when an active ingredient is absorbed through the digestive system.

Accordingly, the present inventors have endeavored to develop a therapeutic agent capable of treating xerostomia in a diabetic patient by administering a therapeutic agent for xerostomia, which occurs as a diabetic complication, in a direct and sustainable fashion, and found that an ethanol extract of Ixeris dentata Nakai significantly increases expression levels of α-amylase, AQP5 and NHE1 whose expression levels have been reduced due to diabetes so that the level of expression of α-amylase, AQP5 and NHE1 is restored to a level similar to those of the healthy model groups when the ethanol extract is sprayed into a sublingual region of a diabetes model rat, and thus a spray formulation to be administered is effective in preventing and treating xerostomia occurring as a diabetic complication by orally spraying the Ixeris dentata Nakai extract of the present invention. Therefore, the present invention has been completed based on these facts.

### SUMMARY OF THE INVENTION

As described above, xerostomia has frequently occurred as a complication of diabetes, but drugs which have been developed as therapeutic agents for xerostomia have drawbacks in that the drugs has a transient effect or cause side effects such as discomfort in the digestive system when orally administered. To solve the above problems, there is a demand for development of a therapeutic agent capable of having a locally significant therapeutic effect on xerostomia.

Therefore, an object of the present invention is to provide an oral spray formulation for preventing and treating xerostomia caused by diabetes.

According to an aspect of the present invention, there is provided an oral spray containing an Ixeris dentata Nakai extract as an active ingredient for preventing and treating xerostomia caused by diabetes.

According to one specific embodiment of the present invention, the Ixeris dentata Nakai extract may be extracted using water, a C₁-C₂ lower alcohol, or a mixture thereof as a solvent, and the lower alcohol may be ethanol or methanol.

According to one specific embodiment of the present invention, the Ixeris dentata Nakai extract may have a concentration of 1 μg/mL to 100 mg/mL.

According to one specific embodiment of the present invention, the Ixeris dentata Nakai extract may promote the secretion of saliva by increasing the expression of one or more proteins selected from the group consisting of α-amylase, aquaporin 5 (AQP5), and a Na⁺/H⁺ ion exchanger (NHE1).

According to one specific embodiment of the present invention, the oral spray may inhibit side effects in the digestive system when the Ixeris dentata Nakai extract is locally administered.

According to another aspect of the present invention, there is provided a quasi-drug containing the Ixeris dentata Nakai extract as an active ingredient for preventing and treating xerostomia caused by diabetes.

According to still another aspect of the present invention, there is provided a quasi-drug containing the Ixeris dentata Nakai extract as an active ingredient for preventing and improving an oral disease caused by oral bacteria.

According to one specific embodiment of the present invention, the quasi-drug may include any one formulation selected from the group consisting of a mouth freshener, mouthwash, an oral ointment, and toothpaste.

According to one specific embodiment of the present invention, the quasi-drug may have an anti-bacterial activity against an oral bacterium which lives in the mouth. In this case, the oral bacterium may include one or more selected from the group consisting of Streptococcus mutans, Veillonella dispar, and Fusobacterium nucleatum.

According to yet another aspect of the present invention, there is provided a method of preventing or treating xerostomia caused by diabetes, which includes spraying an Ixeris dentata Nakai extract into a mouth of a subject in need thereof.

According to one specific embodiment of the present invention, the Ixeris dentata Nakai extract may be extracted using water, a C₁-C₂ lower alcohol, or a mixture thereof as a solvent.

According to one specific embodiment of the present invention, the Ixeris dentata Nakai extract may have a concentration of 1 μg/mL to 100 mg/mL.

According to one specific embodiment of the present invention, the Ixeris dentata Nakai extract may promote the secretion of saliva by increasing the expression of one or more proteins selected from the group consisting of α-amylase, aquaporin 5 (AQP5), and a Na⁺/H⁺ ion exchanger (NHE1).

According to one specific embodiment of the present invention, the method may inhibit side effects in the digestive system when the Ixeris dentata Nakai extract is locally administered.

According to yet another aspect of the present invention, there is provided a method of preventing or treating an oral disease caused by oral bacteria, which includes administering an Ixeris dentata Nakai extract into a mouth of a subject in need thereof.

According to one specific embodiment of the present invention, the Ixeris dentata Nakai extract may have an anti-bacterial effect against one or more oral bacteria selected from the group consisting of Streptococcus mutans, Veillonella dispar, and Fusobacterium nucleatum, all of which live in the mouth.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a schematic diagram showing a flowchart of an experiment after streptozotocin (STZ) is administered to a rat to induce diabetes for use as a diabetes model animal in the present invention;

FIGS. 2A-2D show results of determining an effect of an Ixeris dentata Nakai extract on the improvement of saliva secretion capacity in a diabetes-induced animal model:

FIG. 2A is a diagram showing secretion quantities of saliva in rat groups according to Comparative Examples 1, 2 and 3 and Example 1 of the present invention;

FIG. 2B is a diagram showing secretion rates of saliva in the rat groups according to Comparative Examples 1, 2 and 3 and Example 1 of the present invention;

FIG. 2C is a diagram showing weights of submandibular gland tissues obtained from the rat groups according to Comparative Examples 1, 2 and 3 and Example 1 of the present invention; and

FIG. 2D is a diagram showing total concentrations of proteins in the submandibular gland tissues obtained from the rat groups according to Comparative Examples 1, 2 and 3 and Example 1 of the present invention;

FIG. 3 is a diagram showing that submandibular gland tissues are not restored and improved by the Ixeris dentata Nakai extract in the diabetes-induced animal model;

FIGS. 4A-4D show results of determining an increase in the expression of amylase by the Ixeris dentata Nakai extract in submandibular gland tissues in the diabetes-induced animal model:

FIG. 4A is a diagram showing proteins whose expression levels are changed in the saliva in a diabetes model in which mice are treated with the Ixeris dentata Nakai extract;

FIG. 4B is a diagram showing that a level of expression of α-amylase in submandibular gland tissues is increased in the diabetes model in which mice are treated with the Ixeris dentata Nakai extract;

FIG. 4C is a diagram showing that a level of expression of α-amylase in the saliva is increased in the diabetes model in which mice are treated with the Ixeris dentata Nakai extract; and

FIG. 4D is a diagram showing, through immunohistologic staining, that the level of expression of α-amylase in the submandibular gland tissues is restored in the diabetes model in which mice are treated with the Ixeris dentata Nakai extract;

FIGS. 5A-5B show expression of aquaporin 5 (AQP5) by the Ixeris dentata Nakai extract in submandibular gland tissues in a diabetes animal model:

FIG. 5A shows results of determining, through Western blotting, that AQP5 is increasingly expressed in the submandibular gland tissue in a diabetes model in which mice are treated with the Ixeris dentata Nakai extract; and

FIG. 5B shows results of determining, through immunofluorescent staining, that AQP5 is increasingly expressed in the submandibular gland tissue in the diabetes model in which mice are treated with the Ixeris dentata Nakai extract;

FIG. 6 shows results of determining that α-amylase and AQP5 are co-expressed by the Ixeris dentata Nakai extract in the diabetes animal model;

FIG. 7 shows results of confirming an effect of the Ixeris dentata Nakai extract on an increase in the level of expression of an ion transport protein in the diabetes animal model;

FIGS. 8A-8B show results of confirming an anti-bacterial activity of the Ixeris dentata Nakai extract of the present invention against Streptococcus nutans which is an oral bacterium:

FIG. 8A shows experimental results of confirming an MIC value of the Ixeris dentata Nakai extract in a medium on which the strain is plated at 1×10⁴ CFU/mL; and

FIG. 8B shows experimental results of confirming an MIC value of the Ixeris dentata Nakai extract in a medium on which the strain is plated at 2×10³ CFU/mL;

FIG. 9 shows results of confirming an anti-bacterial activity of the Ixeris dentata Nakai extract of the present invention against Veillonella dispar which is an oral bacterium; and

FIG. 10 shows results of confirming an anti-bacterial activity of the Ixeris dentata Nakai extract of the present invention against Fusobacterium nucleatum which is an oral bacterium.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the scope of the invention.

Unless specifically stated otherwise, all the technical and scientific terms used in this specification have the same meanings as what are generally understood by a person skilled in the related art to which the present invention belongs. In general, the nomenclature used in this specification and the experimental methods described below is widely known and generally used in the related art.

As described above, xerostomia has frequently occurred as a complication of diabetes, but drugs which have been developed as therapeutic agents for xerostomia have drawbacks in that the drugs has a transient effect or cause side effects such as discomfort in the digestive system when orally administered. To solve the above problems, there is a demand for development of a therapeutic agent capable of having a locally significant therapeutic effect on xerostomia.

When the Ixeris dentata Nakai extract of the present invention is sublingually sprayed into a sublingual region of the diabetes model animal, expression levels of α-amylase, AQP5 and NHE1 may significantly increase, resulting in an increased secretion quantity and rate of saliva.

Therefore, the present invention provides an oral spray containing the Ixeris dentata Nakai extract as an active ingredient for preventing and treating xerostomia caused by diabetes.

Also, the present invention provides a quasi-drug containing the Ixeris dentata Nakai extract as an active ingredient for preventing and treating xerostomia caused by diabetes.

The Ixeris dentata Nakai extract of the present invention is preferably extracted using water, a C₁-C₂ lower alcohol, or a mixture thereof as a solvent, and the lower alcohol is preferably ethanol or methanol. Specifically, ethanol is most preferred, but the present invention is not limited thereto.

The Ixeris dentata Nakai extract of the present invention is prepared using a preparation method including the following steps, but the present invention is not limited thereto:

1) adding an extraction solvent to Ixeris dentata Nakai to extract an Ixeris dentata Nakai extract;

2) filtering the extract of Step 1); and

3) concentrating the filtered extract of Step 2) under reduced pressure and drying.

In the method, the Ixeris dentata Nakai of Step 1) may be used without limitation as long as the Ixeris dentata Nakai can be cultivated or is commercially available. Roots, stems, leaves or whole plants of Ixeris dentata Nakai may be used as the Ixeris dentata Nakai, but the present invention is not limited thereto.

In the method, a method of extracting the Ixeris dentata Nakai extract may be performed using conventional methods known in the related art, such as filtration, hot water extraction, dip extraction, reflux extraction, and ultrasonic extraction. The Ixeris dentata Nakai extract is preferably extracted by adding the extraction solvent at a volume of 2 to 20 times the dry weight of the Ixeris dentata Nakai. An extraction temperature is preferably in a range of 20 to 80° C., but the present invention is not limited thereto. Also, an extraction time is preferably in a range of 10 to 100 hours, more preferably in a range of 24 to 96 hours, and most preferably 72 hours, but the present invention is not limited thereto.

In the method, the concentrating under reduced pressure in Step 3) is preferably performed using a vacuum evaporator or a rotary vacuum evaporator, but the present invention is not limited thereto. Also, the drying preferably includes decompression drying, vacuum drying, boiling drying, spray drying, or lyophilizing, but the present invention is not limited thereto.

The Ixeris dentata Nakai extract of the present invention may be included in an oral spray at a concentration of 1 μg/mL to 100 mg/mL. Specifically, the Ixeris dentata Nakai extract may be included at a concentration of 50 μg/mL to 100 mg/mL, and most preferably included at a concentration of 1 mg/mL to 80 mg/mL, but the present invention is not limited thereto. For example, the Ixeris dentata Nakai extract may be applied in a concentration range in which the Ixeris dentata Nakai extract may have an effect of significantly improving xerostomia even when sprayed once.

The Ixeris dentata Nakai extract of the present invention may increase the expression of one or more proteins selected from the group consisting of α-amylase, aquaporin 5 (AQPS), and a Na⁺/H⁺ ion exchanger (NHE1). In other words, the secretion of saliva is reduced as submandibular gland tissues and acinar/ductal cells thereof are destroyed in diabetes, but proteins associated with saliva secretion are increasingly expressed without restoring the shape of the destroyed tissues and cells to improve the secretion of saliva when the Ixeris dentata Nakai extract is administered.

The oral spray of the present invention may inhibit side effects in the digestive system by locally administering the Ixeris dentata Nakai extract. Specifically, using the oral spray of the present invention, the Ixeris dentata Nakai extract is preferably sprayed into a mouth of a diabetic patient. More specifically, the oral spray of the present invention is most preferably sprayed into a sublingual (subglossal) region of the diabetic patient, but the present invention is not limited thereto.

The Ixeris dentata Nakai extract of the present invention may be provided in the form of a quasi-drug in addition to the oral spray. In this case, the quasi-drug may include any one formulation selected from the group consisting of a mouth freshener, mouthwash, an oral ointment, and toothpaste.

According to one specific embodiment of the present invention, the present inventors have administered streptozotocin to rats to establish a diabetes model. The present inventors have confirmed that a high level of blood glucose is present in the established diabetes model, and then sublingually sprayed the Ixeris dentata Nakai ethanol extract.

First of all, the present inventors have found that the Ixeris dentata Nakai extract has an effect of improving saliva secretion capacity in a diabetes model, and confirmed that the secretion quantity and rate of saliva are reduced in a diabetes model, but the secretion quantity and rate of saliva are restored to a level of the healthy comparative control when the Ixeris dentata Nakai extract is sprayed. However, it was confirmed that such an effect of improving saliva secretion capacity is not an effect achieved through morphological restoration of the submandibular gland tissues and duct/acinar cells which have been destroyed by diabetes.

Accordingly, the present inventors have expected that a therapeutic effect of the Ixeris dentata Nakai extract on xerostomia caused by diabetes is due to a change in a level of expression of proteins associated with saliva secretion. The present inventors have administered the Ixeris dentata Nakai extract and confirmed a change in the level of expression of amylase in a diabetes model, and found that a level of expression of α-amylase decreases in the diabetes model, but the level of expression of α-amylase is restored to a level of the healthy comparative control when the Ixeris dentata Nakai extract is sublingually sprayed.

Also, the present inventors have administered the Ixeris dentata Nakai extract and confirmed a change in the level of expression of aquaporin 5(AQP5) which is a water channel protein in the diabetes model, and found that a level of expression of AQP5 decreases in the diabetes model, but the level of expression of AQP5 is restored to a level of the healthy comparative control when the Ixeris dentata Nakai extract is sublingually sprayed.

Further, the present inventors have administered the Ixeris dentata Nakai extract and confirmed a change in the level of expression of a Na⁺/H⁺ ion exchanger (NHE1) which is an ion channel protein in the diabetes model, and found that a level of expression of NHE1 decreases in the diabetes model, but the level of expression of NHE1 is restored to a level of the healthy comparative control when the Ixeris dentata Nakai extract is sublingually sprayed.

Accordingly, the present invention provides an oral spray containing the Ixeris dentata Nakai extract as an active ingredient for preventing and treating xerostomia caused by diabetes. When the Ixeris dentata Nakai extract of the present invention is sublingually sprayed into a sublingual region of a diabetes model animal, the expression levels of α-amylase, AQP5 and NHE1 may increase, resulting in an increased secretion quantity and rate of saliva. When the Ixeris dentata Nakai extract is administered using the formulation of the present invention, the Ixeris dentata Nakai extract is sublingually sprayed through the spray formulation, and thus can be directly locally administered to a salivary gland tissue having an activity to induce the secretion of saliva. Therefore, saliva secretion can be induced by rapid absorption of the active ingredient of the Ixeris dentata Nakai extract in the salivary gland tissue without any absorption process through the digestive system, resulting in minimized side effects.

The oral spray formulation of the present invention may be prepared using one or more buffers (for example, saline or PBS), antioxidants, bacteriostats, chelating agents (for example, EDTA or glutathione), fillers, bulking agents, binders, adjuvants (for example, aluminum hydroxide), suspensions, thickening agents, wetting agents, disintegrating agents or surfactants, diluents, or excipients.

Also, lubricants such as magnesium stearate, talc and the like are used in addition to the simple excipients. A liquid preparation for oral spraying includes a suspension, a liquid for internal use, an emulsion, a syrup, etc. In this case, the liquid preparation includes various excipients, for example, a wetting agent, a sweetening agent, a flavoring agent, a preservative, and the like in addition to the generally used simple diluents such as water, liquid paraffin, etc. Also, cross-linked polyvinyl pyrrolidone, agar, alginic acid, sodium alginate, or the like may be optionally added as the disintegrating agent, and the formulation may further include an anti-clumping agent, a lubricant, a wetting agent, a flavoring agent, an emulsifying agent, and a preservative, etc.

The oral spray of the present invention is prepared in the form of an aerosol spray to deliver a drug more conveniently when sprayed from a pressurized pack or a nebulizer. The compound used accordingly may be readily delivered in the form of an aerosol spray from a pressurized pack or a nebulizer using a suitable propellant, for example, dichlorofluoromethane, trichlorofluoromethane, dichlorotetrafluoroethane, carbon dioxide, or other suitable gases. In the case of pressurized aerosol, the dosage unit may be determined by providing a valve configured to deliver an amount of calculated aerosol. For example, a gelatin capsule and cartridge used in an inhaler or an insufflator may be formulated to include a powder mixture of the compound and a suitable powder base such as lactose or starch.

The Ixeris dentata Nakai extract of the present invention may be administered at a pharmaceutically effective dose. In the present invention, the term “pharmaceutically effective dose” refers to an amount sufficient to treat a disease at a reasonable benefit/risk ratio applicable for medical treatment. In this case, a range of the effective dose may be determined depending on the type and severity a disease in a patient, the activity of a drug, the sensitivity to the drug, an administration time, a route of administration, and a secretion rate, a therapeutic period, factors including drugs to be used together, and other factors well known in the field of medicine. The Ixeris dentata Nakai extract of the present invention may be administered as an individual therapeutic agent, or may be administered in combination with other therapeutic agents. In this case, the Ixeris dentata Nakai extract may be administered sequentially or concurrently with conventional therapeutic agents. Also, the Ixeris dentata Nakai extract may be administered as a single dose or multiple doses. That is, the total effective dose of the Ixeris dentata Nakai extract according to the present invention may be administered to a patient as a single dose, or administered according to a fractionated treatment protocol in which multiple doses are administered for a long period of time. By considering all the above factors, it is important to administer the Ixeris dentata Nakai extract at a dose in which the maximum effect can be achieved without any side effects when administered at a minimum dose. Thus, the dose of the Ixeris dentata Nakai extract may be easily determined by those skilled in the related art.

A dose of the Ixeris dentata Nakai extract according to the present invention may vary depending on the weight, age, sex, health condition, and diet of a patient, an administration time, an administration mode, a secretion rate, and the severity of a disease. The Ixeris dentata Nakai extract may be administered once or several times a day so that the daily dose of the Ixeris dentata Nakai extract is preferably in a range of 0.01 to 50 mg, more preferably 0.1 to 30 mg, based on 1 kg of body weight. However, since the daily dose of the Ixeris dentata Nakai extract may vary depending on a route of administration, the severity of obesity, the sex, weight and age of a patient, etc., the dose is not intended to limit the scope of the present invention in any fashion.

In addition, the present invention provides a quasi-drug containing the Ixeris dentata Nakai extract as an active ingredient for preventing and improving an oral disease caused by oral bacteria.

According to one specific embodiment of the present invention, the quasi-drug may include any one formulation selected from the group consisting of a mouth freshener, mouthwash, an oral ointment, and toothpaste.

According to one specific embodiment of the present invention, the quasi-drug may have an anti-bacterial activity against an oral bacterium which lives in the mouth. In this case, the oral bacterium may include one or more selected from the group consisting of Streptococcus mutans, Veillonella dispar, and Fusobacterium nucleatum.

Hereinafter, the present invention will be described in further detail with reference to examples thereof. However, it will be apparent to those skilled in the art that the following examples are just preferred examples for the purpose of illustration only and are not intended to limit the scope of the invention.

### PREPARATIVE EXAMPLE 1

Preparation of Ixeris dentata Nakai Extract

To perform this inventive experiment, an Ixeris dentata Nakai extract was prepared.

Specifically, Ixeris dentata Nakai was collected in March, 2014 in Dangjin, Korea, and used. 1.2 kg of powder of pulverized Ixeris dentata Nakai roots was immersed in 5 L of ethanol, followed by extraction at 35° C. for 3 hours by sonication, and the resulting extract was filtered. A solvent obtained through filtration was concentrated in a vacuum under reduced pressure to obtain 30.5 g (2.5% based on dry weight) of an Ixeris dentata Nakai ethanol extract. The ethanol extract was dissolved in water so that the ethanol extract was adjusted to a desired concentration before use.

### PREPARATIVE EXAMPLE 2

Preparation of Diabetes Model Rat

<2-1> Establishment of Diabetes Model using Streptozotocin

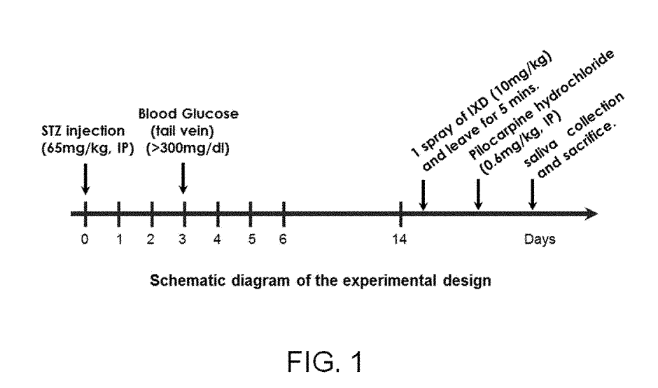

To perform this inventive experiment, a diabetes model rat was prepared (FIG. 1).

First, thirty three 7-week-old male Sprague-Dawley rats weighing 200 to 230 g were purchased, and divided into a total of 4 groups, each group consisting of 7 to 9 rats. The rats in all the groups were bred under general breeding environments (a temperature of 22±2° C., a relative humidity of 55 to 60%, and a day/night lighting cycle of 12 hours), and then adapted to a laboratory environment for a week before use in experiments. Water and feed were freely supplied during the experiments.

Next, 65 mg/kg of streptozotocin (STZ) was dissolved in a 0.1 M citrate buffer (pH 4.5), and intraperitoneally (IP) injected into the bred rats in one group. Thereafter, the rats were bred for 2 weeks to induce diabetes. 3 days after STZ injection, blood was collected from tail arteries of the rats which had been fasted overnight, and a blood glucose value was measured using a glucometer. When the measured blood glucose value was 300 mg/dL, the corresponding rat was diagnosed with diabetes, and then used as a diabetes animal model. In the glucometer, ‘Hi’ indicates that a blood glucose level is 600 mg/dL.

<2-2> Administration of Ixeris dentata Nakai Extract in Diabetes Animal Model

To check a therapeutic effect on mouth dryness in rats in which diabetes had been induced, the Ixeris dentata Nakai extract was administered.

Specifically, the diabetes-induced rats prepared in Preparative Example <2-1> were anesthetized, and the Ixeris dentata Nakai extract (10 mg/50 mL/kg) prepared in [Preparative Example 1] was sprayed into a sublingual region, kept for 5 minutes, and then used as an experimental group in Example 1. In the present invention, the controls prepared to be used together with the experimental group are listed in the following [Table 1].

TABLE 1
Experimental group and controls used in the present invention

| | | Treatment with Ixeris |
|---|---|---|
| | Diabetes induction | dentata Nakai extract |
| Example 1 | STZ injection | Ixeris dentata Nakai extract |
| | | was sprayed |
| Comparative | No diabetes induction | Water was sprayed |
| Example 1 | | |
| Comparative | No diabetes induction | Ixeris dentata Nakai extract |
| Example 2 | | was sprayed |
| Comparative | STZ injection | Water was sprayed |
| Example 3 | | |

### EXPERIMENTAL EXAMPLE 1

Confirmation of Effect of Ixeris dentata Nakai extract on Improvement of Saliva Secretion Capacity in Diabetes-Induced Animal Model

To determine whether the Ixeris dentata Nakai extract of the present invention has a therapeutic effect on mouth dryness caused by diabetes, it was confirmed that the Ixeris dentata Nakai extract had an effect of improving saliva secretion capacity when the Ixeris dentata Nakai extract was administered.

<1-1> Comparison of Total Secretion Quantities of Saliva

0.6 mg/kg of pilocarpine hydrochloride was IP administered to the rats in the rat groups of Example 1, and Comparative Examples 1, 2 and 3, and kept for 5 minutes to induce the rats to easily secrete saliva. The secreted saliva was allowed to be absorbed into cotton wool for approximately 30 minutes. Thereafter, the weight of the cotton wool was measured, and converted into milliliter units (mL). The flow rate of saliva was calculated as a quantity (μL) of saliva secreted per unit time (minute). In addition, the total amount of proteins in the secreted saliva was determined using a Bradford assay.

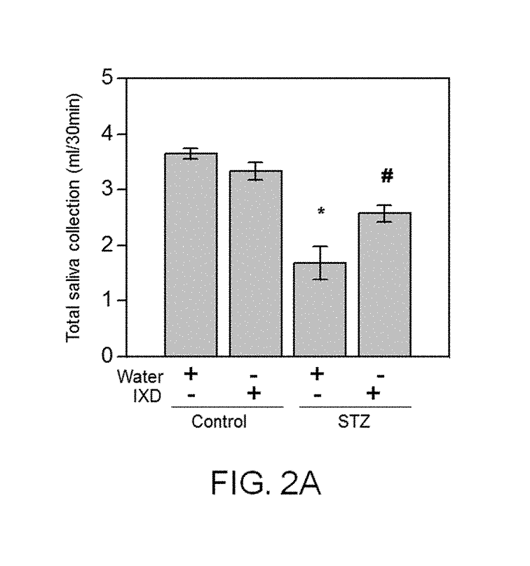

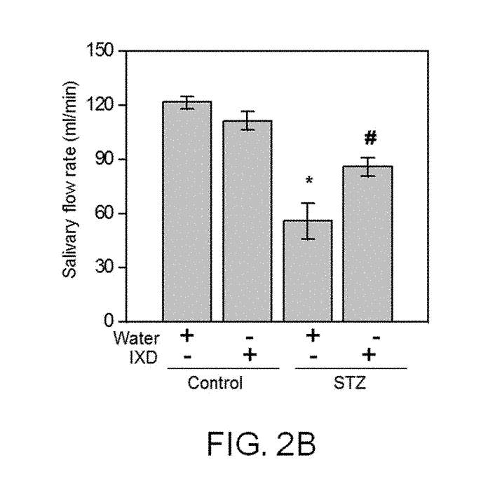

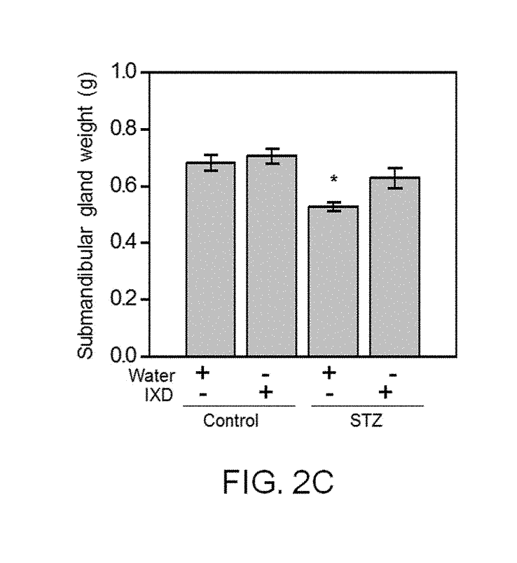

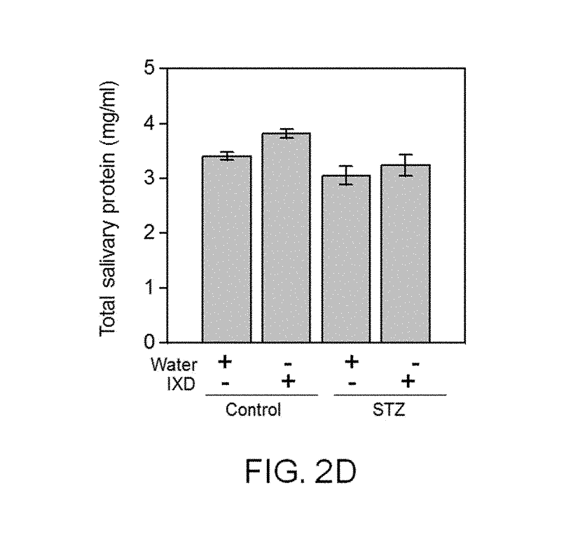

As a result, it was revealed that there was no significant difference in secretion quantities and rates of saliva in the healthy rat groups in which diabetes was not induced (Comparative Examples 1 and 2), as shown in FIGS. 2A and 2B. However, it was revealed that the total secretion quantities and rates of saliva were remarkably reduced in the diabetic rats (Comparative Example 3), and that the secretion quantities and rates of saliva were remarkably increased when the Ixeris dentata Nakai extract was sprayed (Example 1) (FIGS. 2A and 2B). Also, it was confirmed that there was no significant difference in the total amount of proteins in the saliva from all the rat groups, as shown in FIG. 2D.

<1-2> Comparison of Weights of Submandibular Glands

After the saliva secretion capacities were compared, the rats were sacrificed to remove submandibular glands on both sides thereof. The weights of the removed submandibular glands were measured. Thereafter, one of the submandibular glands was immediately frozen, and the other submandibular gland was immersed in a 3.7% formalin solution, and then used as a sample for histological inspection.

As a result, it was confirmed that the weights of the submandibular glands of the diabetic rats (Comparative Example 3) were significantly reduced, compared to those of the healthy rat groups (Comparative Examples 1 and 2) in which diabetes was not induced, as shown in FIG. 2C. However, it was confirmed that, when the Ixeris dentata Nakai extract was sprayed, the weights of the submandibular glands were significantly increased to a level similar to those of the healthy rats, indicating an increase in secretion of saliva.

From the results, it was confirmed that the Ixeris dentata Nakai extract of the present invention had an effect of improving mouth dryness caused by diabetes since the Ixeris dentata Nakai extract induced saliva secretion while inhibiting saliva secretion dysfunction.

### EXPERIMENTAL EXAMPLE 2

Confirmation of Effect of Ixeris dentata Nakai Extract on Improvement of Submandibular Gland Tissue in Diabetes-Induced Animal Model

Since the Ixeris dentata Nakai extract had an effect of improving the saliva secretion capacity in the diabetes animal model, it was determined whether such an improvement effect is an effect achieved through restoration of saliva secretion-associated tissues.

Specifically, after the saliva secretion capacities of the rat groups of Example 1, and Comparative Examples 1, 2 and 3 were compared, the rats were sacrificed to remove submandibular glands on both sides thereof. The removed submandibular gland tissues were embedded in paraffin, fixed, and then microtomed into pieces having a thickness of 5 μm. The microtomed pieces were deparaffinized, and dehydrated with alcohol. Thereafter, the tissues were washed with water, and stained with Mayer's hematoxylin and 1% eosin. After the staining, the tissues were again dehydrated with alcohol, and then wiped with xylene to complete samples. The completed samples were observed under an optical microscope.

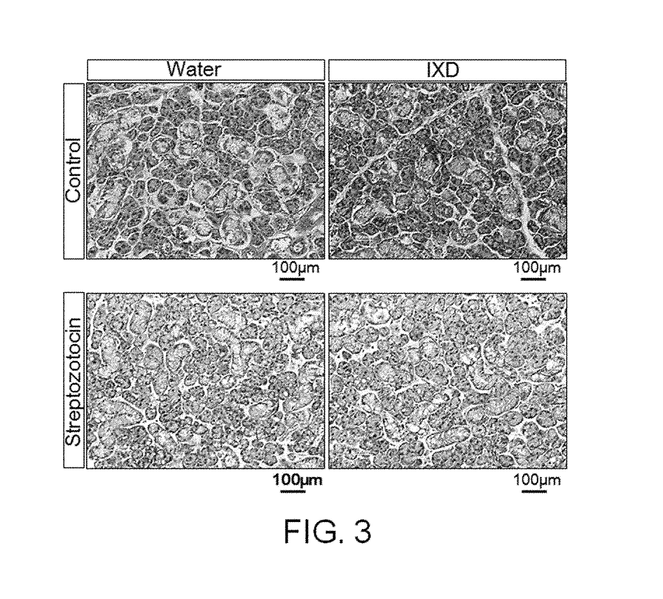

As a result, it was confirmed that a large number of acinar cells were present and duct cells were highly conserved regardless of the administration of the Ixeris dentata Nakai extract in the case of the healthy rat groups (Comparative Examples 1 and 2), as shown in FIG. 3. On the other hand, it was confirmed that the acinar cells were destroyed and the duct cells were irregularly distributed in the submandibular gland tissues of the diabetic rats (Comparative Example 3). The Ixeris dentata Nakai extract had no effect of restoring the destroyed submandibular gland tissues when the Ixeris dentata Nakai extract was sublingually sprayed into the diabetic rats in which the tissues were destroyed. From these results, even when the cell tissues of the submandibular glands were destroyed due to diabetes, it was confirmed that the saliva secretion was able to be significantly improved by the Ixeris dentata Nakai extract when the Ixeris dentata Nakai extract was sublingually sprayed.

### EXPERIMENTAL EXAMPLE 3

Confirmation of Increase in Expression of Amylase in Submandibular Gland tissues by Ixeris dentata Nakai extract in Diabetes-Induced Animal Model

<3-1>Confirmation of Proteins whose Expression Levels in Saliva are Changed by Ixeris dentata Nakai Extract in Diabetes-Induced Animal Model

It was confirmed that, when the salivary gland tissues were destroyed by diabetes, the saliva secretion capacity was able to be improved without the Ixeris dentata Nakai extract exhibiting an effect of improving the destroyed tissues. Based on this fact, the presence of proteins whose expression levels increased in the tissues was confirmed to check a more specific effect.

Specifically, the saliva was collected from the rat groups of Example 1, and Comparative Examples 1, 2 and 3, and the total amount of proteins in the saliva was determined using a Bio-Rad protein assay kit. Thereafter, SDS-PAGE and then Coomassie-Brilliant Blue staining were performed to confirm proteins bands.

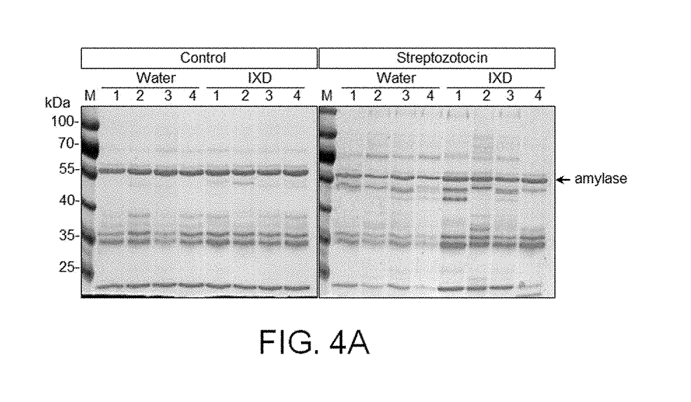

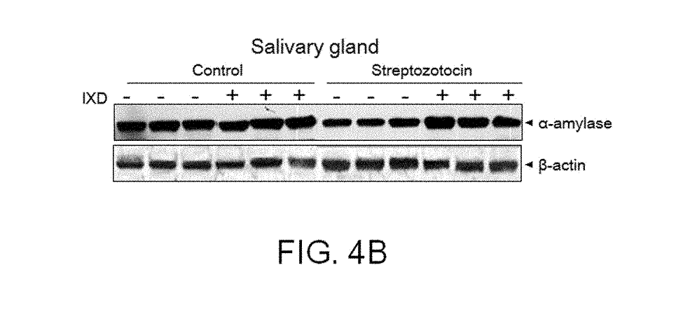

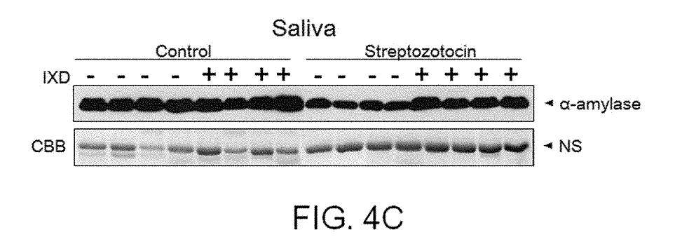

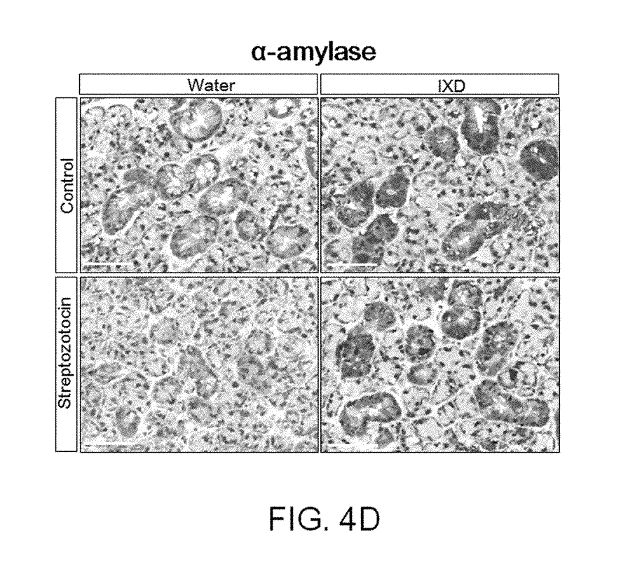

As a result, it was confirmed that the expression of the proteins was not significantly changed regardless of the administration of the Ixeris dentata Nakai extract in the case of the healthy rat groups (Comparative Examples 1 and 2), whereas it was confirmed that the expression of a protein having a molecular weight of approximately 55 kDa was significantly reduced in the saliva from the diabetic rats (Comparative Example 3), as shown in FIG. 4A. On the other hand, it was confirmed that the expression of the 55-kDa protein whose expression had been reduced significantly increased in the experimental group of Example 1 when the Ixeris dentata Nakai extract was sublingually sprayed.

<3-2>Confirmation of Increase in Expression Level of Amylase by Ixeris dentata Nakai Extract in Diabetes-Induced Animal Model

The proteins whose expression had been increased by the Ixeris dentata Nakai extract in the diabetes model were specifically confirmed.

To identify the proteins whose expression levels significantly increased after SDS-PAGE in Experimental Example <3-1>, Western blotting was performed. From the SDS-PAGE results, proteins developed on a membrane were transferred to polyvinylidene fluoride (PVDF), and the membrane was blocked with a Tris buffer solution (0.137 M NaCl, 0.025 M Tris, pH 7.4) (T-BST) including 5% skimmed milk and 0.1% Tween-20. After the blockage, the membrane was treated with an anti-amylase antibody as a primary antibody, incubated overnight at 4° C., and then washed with T-BST. Thereafter, the membrane was treated with an anti-mouse antibody as a secondary antibody, and incubated at room temperature for an hour, and bands of the proteins were visualized using an enhanced chemiluminescence (ECL) reagent. The membrane was exposed to an imaging film, and then developed using a Kodak X-OMAT 1000A processor. To check a change in expression level of amylase in the submandibular gland tissue as well as the saliva, submandibular gland tissues were removed from the rats sacrificed after the collection of saliva, and homogenized with a homogenizer to obtain a tissue extract. Then, Western blotting was performed on the tissue extract in the same manner.

As a result, it was confirmed that there was no significant change in the expression level of α-amylase regardless of the administration of the Ixeris dentata Nakai extract in the case of the healthy rat groups (Comparative Examples 1 and 2), but the expression of α-amylase in the saliva and submandibular gland tissues of the diabetic rat (Comparative Example 3) was significantly reduced, as shown in FIGS. 4B and 4C. On the other hand, it was confirmed that, when the Ixeris dentata Nakai extract was sublingually sprayed, the expression of α-amylase whose expression had been reduced was significantly increased in the experimental group of Example 1, and thus the expression level of α-amylase increased approximately twofold in all the saliva and submandibular gland tissues, compared to the control of Comparative Example 3.

<3-3>Confirmation of Expression of Amylase in Submandibular Gland Tissue by Ixeris dentata Nakai Extract in Diabetes Animal Model

It was confirmed that the expression level of amylase in the saliva and submandibular gland tissues in the diabetes model was increased by the Ixeris dentata Nakai extract. To confirm the results in a more specific manner, the expression and site of α-amylase were determined through immunohistochemical staining.

Specifically, the rats in the rat groups of Example 1, and Comparative Examples 1, 2 and 3 were sacrificed to remove submandibular glands on both sides thereof. The removed tissues were embedded in paraffin, fixed, and then microtomed into pieces having a thickness of 5 μm. The microtomed pieces were deparaffinized, and dehydrated. To find a suitable antibody, slides carrying the tissue pieces were immersed in a 1× target retrieval solution (pH 6.0), and stored at 125° C. for 20 minutes. Thereafter, the tissues were stored in iced water for 30 minutes, and then washed with deionized water for 5 minutes. The washed tissues were again treated with a phosphate butter including hydrogen peroxide, 15 mmol/L NaN₃ and a surfactant for 10 minutes to block an intracellular peroxidase activity. After the blockage, the tissues were immersed twice in a 1× T-BST buffer for 5 minutes, and immersed in PBS (including 0.25% casein) to block non-specific binding to proteins, and then kept at room temperature for 10 minutes. Subsequently, the tissues were treated with an anti-amylase antibody (1:100 dilution, Santa Cruz Biotechnology) as a primary antibody, and incubated overnight at 4° C. After the incubation, the tissue pieces were washed with a TBST buffer, and then treated with an anti-mouse antibody as a secondary antibody at room temperature for an hour. Then, the tissue pieces were again washed with TBST supplemented with AEC substrate chromogen (DAKO), and then re-washed with deionized water to prepare the tissues. The prepared tissues were finally stained with Harris hematoxylin to confirm an expression site and an expression level of α-amylase in the tissues.

As a result, it was confirmed that the expression level of α-amylase was maintained to a sufficient extent regardless of the administration of the Ixeris dentata Nakai extract, and the acinar and duct cells were highly conserved in the case of the healthy rat groups (Comparative Examples 1 and 2), as shown in FIG. 4D. On the other hand, it was confirmed that the acinar and duct cells were destroyed in the submandibular gland tissues of the diabetic rats (Comparative Example 3), and the expression of α-amylase in granular convoluted tubule cells was reduced. However, it was confirmed that the expression of α-amylase in the submandibular gland tissues was significantly restored after the Ixeris dentata Nakai extract was sublingually sprayed in the experimental group of Example 1.

From the results, it was confirmed the Ixeris dentata Nakai extract of the present invention had no effect on the shape of the submandibular gland tissues and cells, but the expression of α-amylase was significantly restored in the submandibular gland tissues and cells which had been destroyed by diabetes.

### EXPERIMENTAL EXAMPLE 4

Confirmation of Increase in Expression of Aquaporin 5 (AQP5) in Submandibular Gland Tissues by Ixeris dentata Nakai Extract in Diabetes-Induced Animal Model

As a protein functionally associated with the saliva secretion in acinar cells of a submandibular gland tissue under normal physiological conditions, aquaporin (AQP5) is known to play a role in transporting water into the lumen. Therefore, the present inventors have examined whether the Ixeris dentata Nakai extract has a regulatory effect on a decrease in expression of AQP5, which is one cause by which xerostomia is induced due to a decrease in saliva secretion caused by diabetes.

First, the rats in the rat groups of Example 1, and Comparative Examples 1, 2 and 3 were sacrificed to remove submandibular glands on both sides thereof. One of the removed submandibular gland tissues was homogenized with a homogenizer to obtain an extract, which was then subjected to Western blotting. An anti-AQP5 antibody (1:1000 dilution, Abcam) was used as a primary antibody for Western blotting, and an anti-goat antibody was used as a secondary antibody to determine a level of expression of AQP5 in the tissues.

The other of the removed submandibular gland tissues was embedded in paraffin, and then subjected to an immunohistochemical assay using an anti-AQP5 antibody (1:100 dilution, Abcam) as a primary antibody and an anti-rabbit antibody as a secondary antibody, thereby determining an expression site and an expression level of AQP5 in the tissues.

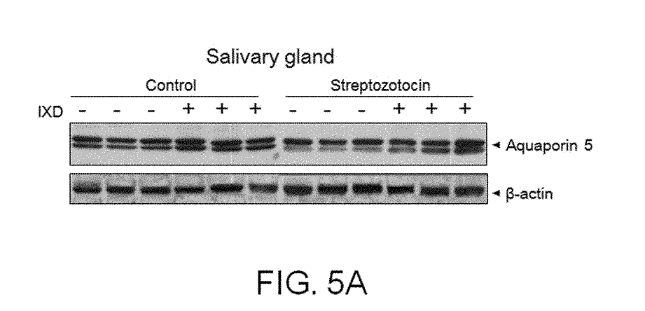

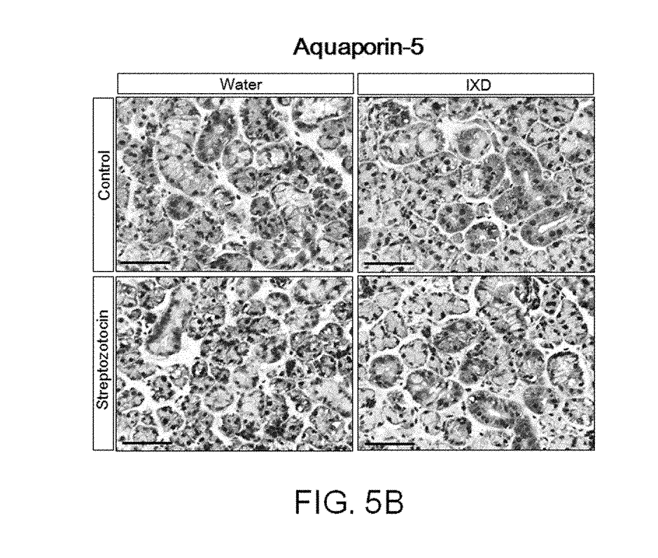

As a result, it was confirmed that AQP5 was expressed at a high expression level regardless of the administration of the Ixeris dentata Nakai extract in the case of the healthy rat groups of Comparative Examples 1 and 2, as shown in FIGS. 5A and 5B. On the other hand, it was confirmed through a Western blotting assay that the expression level of AQP5 was significantly reduced in the diabetic rats of Comparative Example 3.

Based on previous studies, it was known that the expression of AQP5 is observed at various positions of the submandibular glands, and the secretion quantity of saliva may be regulated depending on an increase or decrease in the expression level of AQP5 since AQP5 is located at an apical membrane, a basal membrane and a lateral membrane in the acinar cells. It was reported that AQP5 is partially expressed in duct cells as well as the acinar cells. Thus, it was confirmed that the expression of AQP5 was uniformly distributed in the tissues from the rats of Comparative Examples 1 and 2 of the present invention. On the other hand, it was confirmed that the expression level of AQP5 was highly reduced in the diabetic rat of Comparative Example 3.

However, it was confirmed that the expression level of AQP5 in the tissues significantly increased in the rat group of Example 1 in which the Ixeris dentata Nakai extract was sprayed in the diabetes model, and restored to the expression level of AQP5 in the tissues from the rats of Comparative Examples 1 and 2. For the expression site, it was confirmed that AQP5 was consistently expressed at a high expression level in the duct and acinar cells secreting saliva. From the results, it was confirmed that the Ixeris dentata Nakai extract of the present invention restored the expression level of AQP5 whose expression level had decreased as the submandibular gland tissues were destroyed by diabetes, and thus had an effect of increasing a secretion rate of saliva since AQP5 increased the permeability of water through acinar cell membranes to enable smooth transportation between isotonic solutions.

### EXPERIMENTAL EXAMPLE 5

Determination of Local Expression of α-amylase and AQP5 by Ixeris dentata Nakai Extract in Diabetes Animal Model

Since it was confirmed that the Ixeris dentata Nakai extract had an effect of significantly improving saliva secretion when the Ixeris dentata Nakai extract was locally sprayed into a sublingual region of a rat in the diabetes model, it was confirmed once more whether the Ixeris dentata Nakai extract had an effect of increasing the expression levels of α-amylase and AQP5 at the same time.

Specifically, the rats in the rat groups of Example 1, and Comparative Examples 1, 2 and 3 were sacrificed to remove submandibular glands on both sides thereof. The removed tissues were embedded in paraffin, fixed, and then microtomed into pieces having a thickness of 5 μm. The microtomed pieces were deparaffinized, and dehydrated. Thereafter, the tissue pieces were incubated in a blocking buffer (Dako) for 10 minutes, an anti-amylase antibody (1:200 dilution, Santa Cruz Biotechnology) was added thereto, and the tissue pieces were incubated overnight at 4° C. The next day, the tissue pieces were washed, and then treated for an hour with an FITC-conjugated anti-mouse antibody (1:300 dilution, Sigma) as a secondary antibody. The tissue pieces were washed again, treated with a rabbit-derived anti-AQP5 antibody (1:200 dilution, Abcam), and then incubated overnight at 4° C. Further, the tissues were washed again, treated with a TRITC-conjugated anti-rabbit antibody (1:300 dilution), and then incubated for an hour. Subsequently, the expression of intracellular proteins was observed under an immuno-microscope using FITC and TRITC channels.

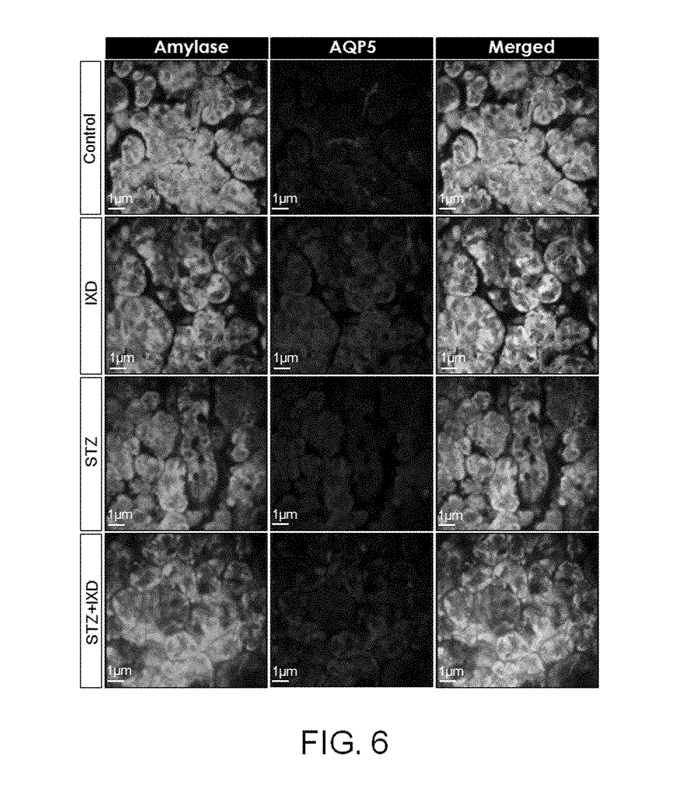

As a result, it was confirmed that all the amylase and AQP5 were sufficiently expressed regardless of the administration of the Ixeris dentata Nakai extract in the case of Comparative Examples 1 and 2, as shown in FIG. 6. However, it was revealed that the expression levels of both amylase and AQP5 tended to decrease in the diabetic rats of Comparative Example 3. It was confirmed that the expression of amylase and AQP5 increased and the expression sites of amylase and AQP5 were similar in the rat group of Example 1 in which the Ixeris dentata Nakai extract was administered to the diabetic rats, indicating that the co-expression of amylase and AQP5 locally increased.

From the results, it was confirmed that, when the Ixeris dentata Nakai extract was sprayed into sublingual regions of rats in a diabetes model, the expression of amylase locally increased, and a water channel protein increased due to the presence of AQP5 whose expression level had increased so as to continuously secrete saliva, which aided in improving xerostomia.

### EXPERIMENTAL EXAMPLE 6

Confirmation of Effect of Ixeris dentata Nakai Extract on Increase in Expression Level of Ion Channel Protein in Diabetes Animal Model

Since it was confirmed that the expression of the water channel protein AQP5 significantly increased when the Ixeris dentata Nakai extract was locally sprayed into sublingual regions of the rats in the diabetes model, it was also confirmed whether the expression level of the ion channel protein was changed.

Specifically, the rats in the rat groups of Example 1, and Comparative Examples 1, 2 and 3 were sacrificed to remove submandibular glands on both sides thereof. The removed tissues were embedded in paraffin, fixed, and then subjected to an immunohistochemical assay using an anti-NHE1 antibody (1:100 dilution, Santa Cruz Biotechnology) as a primary antibody and an anti-rabbit antibody as a secondary antibody so as to determine an expression site and an expression level of the NHE1 protein in the tissues.

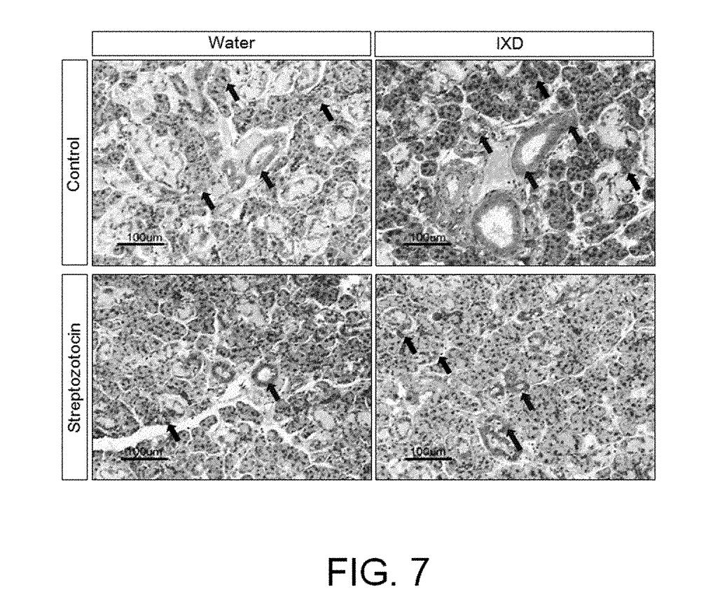

As a result, it was confirmed that the NHE1 protein was expressed at a sufficient expression level regardless of the administration of the Ixeris dentata Nakai extract, and expressed at a higher level in the duct cells than in the acinar cells in the healthy rat groups (Comparative Examples 1 and 2), as shown in FIG. 7. On the other hand, it was confirmed that the expression level of NHE1 was reduced in the submandibular gland tissues of the diabetic rats (Comparative Example 3). However, in the rat group of Example 1, it was confirmed that the expression of NHE1 was significantly increased in both the duct and acinar cells of the submandibular gland tissues when treated with the Ixeris dentata Nakai extract. From the fact that the local expression of NHE1 in the duct cells increased, it was confirmed that the Ixeris dentata Nakai extract increased the expression of NHE1 in the submandibular gland tissues of the diabetic rats to aid in absorption of NaC1 by the duct cells while increasing the secretion quantity of saliva from the acinar cells.

### EXPERIMENTAL EXAMPLE 7

Confirmation of Anti-bacterial Activity of Ixeris dentata Nakai Extract Against Oral Bacteria

To determine whether the Ixeris dentata Nakai extract of the present invention has a significant anti-bacterial activity against the oral bacteria, the anti-bacterial activities against Streptococcus mutans, Veillonella dispar and Fusobacterium nucleatum), all of which cause inflammation in the mouth, were confirmed.

Specifically, each of the strains Streptococcus mutans KCOM1075, Veillonella dispar KCOM2195 and Fusobacterium nucleatum KCOM2927 was incubated. Each of the incubated strains was diluted to a concentration of 1×10³, 2×10³ and 1×10⁴ cells/mL, and dispensed into a 96-well microtiter plate. 100 μL of the strain prepared through the dilution was dispensed into an agar medium, and the Ixeris dentata Nakai extract prepared in [Preparative Example 1] was inoculated and plated. The plated medium was incubated at 37° C. for 16 hours in an incubator, and incubated again for another 16 hours to count the resulting colonies. An experimental group in which the strain alone was dispensed and the Ixeris dentata Nakai extract was not plated was used as the negative control.

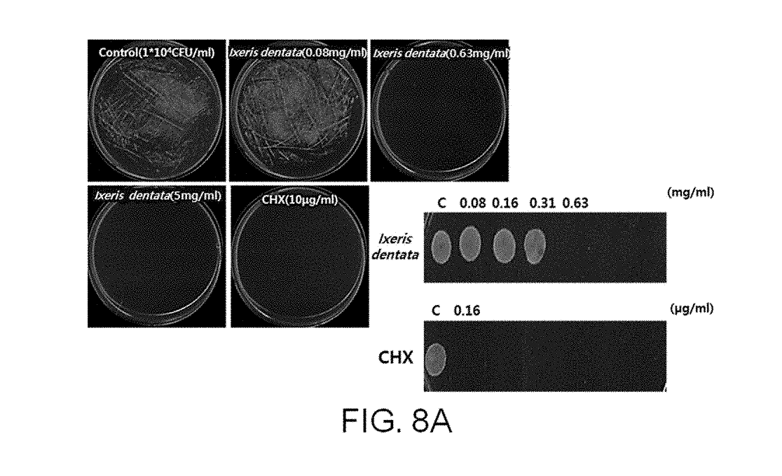

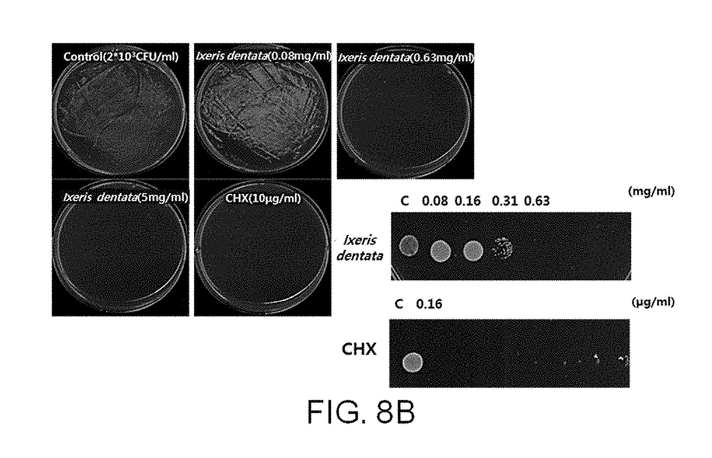

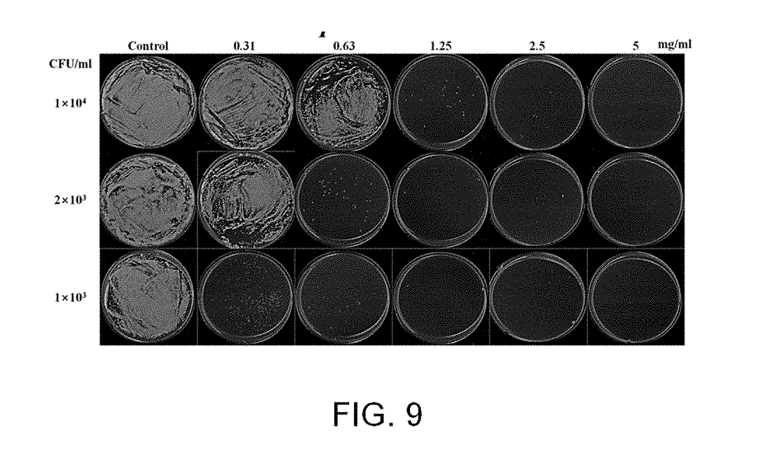

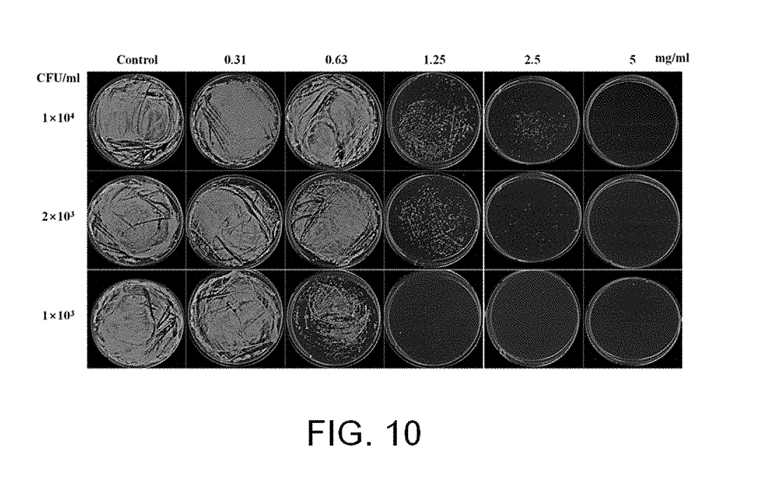

As a result, it was confirmed that the Ixeris dentata Nakai extract of the present invention has a significant anti-bacterial activity against oral pathogenic microbes causing inflammation in the mouth, and also confirmed that the Ixeris dentata Nakai extract had a minimum inhibitory concentration (MIC) of 0.31 to 0.63 mg/mL against all the strains when inoculated at a density of 1 x10³ cells/mL, as shown in FIGS. 8A, 8B, 9 and 10 and listed in the following [Table 2]. It was confirmed that the MIC value was in a range of 0.31 to 1.25 mg/mL even when the strain was dispensed at densities of 2×10³ and 1×10⁴ cells/mL.

TABLE 2
Confirmation of anti-bacterial activity of Ixeris dentata
Nakai extract against oral bacteria

| | MIC (mg/mL) | | |
|---|---|---|---|
| Strain name | 1 × 10³ | 2 × 10³ | 1 × 10⁴ |
| Streptococcus mutans (KCOM 1075) | <0.31 | 0.31 | 0.63 |
| Veillonella dispar (KCOM 2195) | 0.31 | 0.63 | 1.25 |
| Fusobacterium nucleatum (KCOM 2927) | 0.63 | 1.25 | 1.25 |

Therefore, the present invention provides an oral spray containing the Ixeris dentata Nakai extract as an active ingredient for preventing and treating xerostomia caused by diabetes.

The secretion of saliva is reduced as cells from a submandibular gland tissue of a diabetes model animal are destroyed. However, when the Ixeris dentata Nakai extract of the present invention is sublingually sprayed into a sublingual region of the diabetes model animal, expression levels of α-amylase, AQPS and NHE1 can significantly increase, resulting in an increased secretion quantity and rate of saliva. When the Ixeris dentata Nakai extract is administered using the formulation of the present invention, the Ixeris dentata Nakai extract is sublingually sprayed through the spray formulation, and thus can be directly locally administered to a salivary gland tissue having an activity to induce the secretion of saliva. Therefore, saliva secretion can be induced by rapid absorption of the active ingredient of the Ixeris dentata Nakai extract in the salivary gland tissue without any absorption process through the digestive system, resulting in minimized side effects.

It will be apparent to those skilled in the art that various modifications can be made to the above-described exemplary embodiments of the present invention without departing from the scope of the invention. Thus, it is intended that the present invention covers all such modifications provided they come within the scope of the appended claims and their equivalents.

1. A method of preventing or treating xerostomia caused by diabetes, comprising:
oral spraying an Ixeris dentata Nakai extract into a mouth of a subject in need thereof.

2. The method of claim 1, wherein the Ixeris dentata Nakai extract is extracted using water, a C₁-C₂ lower alcohol, or a mixture thereof as a solvent.

3. The method of claim 2, wherein the lower alcohol is ethanol or methanol.

4. The method of claim 1, wherein the Ixeris dentata Nakai extract has a concentration of 1 μg/mL to 100 mg/mL.

5. The method of claim 1, wherein the Ixeris dentata Nakai extract promotes secretion of saliva by increasing expression of one or more proteins selected from the group consisting of α-amylase, aquaporin 5 (AQPS), and a Na⁺/H⁺ ion exchanger (NHE1).

6. The method of claim 1, wherein the oral method inhibits side effects in a digestive system when the Ixeris dentata Nakai extract is locally administered.

7. A method of preventing or treating xerostomia caused by diabetes, comprising:
oral administering an Ixeris dentata Nakai extract into a mouth of a subject in need thereof.

8. A method of preventing or treating an oral disease caused by oral bacteria, comprising:
oral administering an Ixeris dentata Nakai extract into a mouth of a subject in need thereof.

9. The method of claim 7, wherein the oral administering further comprises administering any one formulation selected from the group consisting of a mouth freshener, mouthwash, an oral ointment, and toothpaste.

10. The method of claim 7, wherein the Ixeris dentata Nakai extract has an anti-bacterial effect against one or more oral bacteria selected from the group consisting of Streptococcus mutans, Veillonella dispar, and Fusobacterium nucleatum, all of which live in the mouth.

11. The method of claim 8, wherein the oral administering further comprises administering any one formulation selected from the group consisting of a mouth freshener, mouthwash, an oral ointment, and toothpaste.

12. The method of claim 8, wherein the Ixeris dentata Nakai extract has an anti-bacterial effect against one or more oral bacteria selected from the group consisting of Streptococcus mutans, Veillonella dispar, and Fusobacterium nucleatum, all of which live in the mouth.
